(19) United States
(12) Patent Application Publication
(10) Pub. No.: US 2005/0096591 A1
(43) Pub. Date: May 5, 2005

(54) Inflation adaptor with axial insertion block

(75) Inventors: Gregory R. Dion, Tucson, AZ (US); Ghaleb A. Sater, Lynnfield, MA (US); David J. Sauvageau, Methuen, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC.

(21) Appl. No.: 10/702,008

(22) Filed: Nov. 5, 2003

(51) Int. Cl. A61M 29/00

(52) U.S. Cl. 604/99.01

(57) ABSTRACT

A system for treating a vascular condition, including a hollow guidewire having a central lumen, a core wire received in the central lumen of the hollow guidewire and partially extended from a proximal end of the hollow guidewire, an inflatable balloon attached proximate a distal end of the hollow guidewire, a detachable inflation adaptor, and a clamping device. The inflation adaptor includes an axial insertion block having a funnel-shaped receiver to guide the extended core wire into the axial insertion block. The clamping device is axially aligned with the receiver. The extended core wire and the hollow guidewire are translated relative to each other to control a flow of inflation fluid into the inflatable balloon while the clamping device is in a clamped position. An inflation adaptor and a method of operation are also disclosed.

### FIELD OF THE INVENTION

This invention relates generally to balloon catheters and guidewire deployment of catheter-based treatment tools. More specifically, the invention relates to an axially loaded inflation adaptor for an occlusion catheter.

### BACKGROUND OF THE INVENTION

In certain medical treatment procedures, multiple treatment catheters are introduced over and removed from a guidewire. Guidewires are used conventionally to guide the insertion of various medical instruments such as catheters to a desired treatment location within a patient's vasculature. In a typical procedure, the clinician forms an access point for the guidewire by creating an opening in a peripheral blood vessel, such as the femoral artery. The flexible guidewire is introduced through the opening into the peripheral blood vessel, and advanced by the clinician through the patient's blood vessels until the guidewire extends across the vessel segment to be treated. Various treatment catheters, such as a balloon dilatation catheter for a percutaneous transluminal coronary angioplasty, are inserted over the guidewire and similarly advanced through vasculature until they reach the treatment site. The guidewire may be hollow with an inflatable member such as a balloon mounted at its distal end, and an inflation lumen between the inflatable member and an inflation port at its proximal end.

Because treatment catheters may be exchanged over a single hollow guidewire inserted into the body, such hollow guidewires have been designed with detachable, low-profile inflation adaptors and valves that open and close to control the passage of inflation fluid to and from balloons mounted on the guidewires. These balloon guidewires may include a removable inflation manifold or inflation adaptor and an integral valve to maintain the balloon in the inflated state when the manifold is removed. A low-profile catheter valve is advantageous for use with occlusion guidewires, as well as therapeutic or anchorable devices that may have outer diameters of 0.014 inches or smaller. Further details regarding catheter valves, catheter balloons, and inflation adaptors are found in “Low Profile Catheter Valve and Inflation Adaptor,” Zadno-Azizi et al., U.S. patent Publication No. 2002/0133117 published Sep. 19, 2002; “Low Profile Catheter Valve and Inflation Adaptor,” Zadno-Azizi et al., U.S. Pat. No. 6,325,778; and “Guidewire Inflation System,” Zadno-Azizi et al., U.S. Pat. No. 6,050,972, all of which are hereby incorporated by reference in their entirety. Details of various inflation adaptors are found in “Inflation Adaptor and Method of Use,” pending U.S. patent publication 10/348,046 filed Jan. 17, 2003, the contents of which are hereby incorporated by reference in their entirety. An integrated inflation/deflation device for delivery of inflation fluid is described in “Integrated Inflation/Deflation Device and Method,” Bagaoisan, et al., U.S. Pat. No. 6,234,996, the contents of which are hereby incorporated by reference in their entirety.

An exemplary inflation adaptor, which may be attached to a low-profile catheter or a hollow guidewire, provides a fluid-tight chamber for introduction of a pressurized fluid that expands a catheter balloon. The inflation chamber releaseably seals its inflation inlet to the inflation port of an elongate, hollow guidewire to form a fluid passage there between. Fluid is supplied to the inflation port under pressure via the fluid passageway. The inflation adaptor also releaseably grips or clamps portions of the hollow guidewire for sliding operation of a valve that controls the flow of inflation fluid to inflate and deflate a catheter balloon. The adaptor may be detached from the hollow guidewire without deflating the balloon, and the balloon remains inflated until the adaptor is again attached to the catheter, the valve is opened, and the inflation fluid is removed.

A typical inflation adapter has a channel for transversely receiving the hollow guidewire, and includes clips or guides to help align the guidewire within the channel prior to clamping and sealing the inflation adapter about the hollow guidewire. When a hollow guidewire is installed in an exemplary adaptor, an inflation port in the guidewire lies within the fluid-tight inflation chamber of the adaptor. The alignment of the flexible, hollow guidewires and small core wires in the adaptor is critical for a balloon catheter system to work properly. During transverse loading of the fragile hollow guidewire into the inflation adapter, the guidewire may become bent, kinked and/or misaligned with the clamps and seals prior to closing the adapter about the guidewire.

What is required is an improved inflation adaptor that provides faster, easier insertion and alignment of a core wire and a hollow guidewire in the inflation adaptor. The inflation adaptor should allow for easy loading of a core wire and a hollow guidewire into the adaptor while avoiding bending or malformation of the core wire or the hollow guidewire shaft. The inflation adaptor should also provide simple, repeatable positioning of the hollow guidewire within the adapter prior to closing the adapter about the guidewire. A hollow guidewire that is undamaged and correctly positioned within an inflation adapter will provide predictable control of fluid through the hollow guidewire and increased utility and performance of associated medical devices used during the treatment of vascular conditions.

### SUMMARY OF THE INVENTION

One aspect of the invention provides a system for treating a vascular condition including a hollow guidewire having a central lumen, a core wire received in the central lumen of the hollow guidewire and partially extended from a proximal end of the hollow guidewire, an inflatable balloon attached proximate to a distal end of the hollow guidewire, and a detachable inflation adaptor. The detachable inflation adaptor accommodates axial, rather than transverse, loading of a hollow guidewire therein. The detachable inflation adaptor includes an axial insertion block having a funnel-shaped receiver to guide insertion of the extended core wire into the axial insertion block, and a clamping device axially aligned with the receiver. The extended core wire and the hollow guidewire are translated relative to each other to control flow of inflation fluid into the inflatable balloon while the clamping device is in a clamped position.

Another aspect of the invention is an inflation adaptor including a housing, an axial insertion block, and a clamping device. The axial insertion block is positioned within the housing to axially, rather than transversely, receive a core wire that extends from a proximal end of a hollow guidewire. The axial insertion block has a funnel-shaped receiver to guide insertion of the extended core wire into the axial insertion block, and the clamping device is axially aligned with the receiver. The extended core wire and the hollow guidewire are translated relative to each other to control flow of inflation fluid into the hollow guidewire when the clamping device is in a clamped position.

Another aspect of the invention is a method of operating an inflation adaptor. The inflation adaptor is positioned to receive a core wire partially extended from a proximal end of a hollow guidewire. The extended core wire is axially inserted into a funnel-shaped receiver of an axial insertion block. A portion of the extended core wire and the hollow guidewire are clamped. The core wire is translated with respect to the hollow guidewire while in a clamped position, and flow of an inflation fluid through a portion of the hollow guidewire is controlled based on the relative translation.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings, which are not to scale.

### BRIEF DESCRIPTION OF THE DRAWINGS

Various embodiments of the present invention are illustrated by the accompanying figures, wherein:

FIG. 1 is an illustrative view of a system for treating a vascular condition, in accordance with one embodiment of the current invention;

FIG. 2 is an illustrative top view of an inflation adaptor, in accordance with one embodiment of the current invention;

FIG. 3a is a longitudinal cross-sectional view of a portion of a plug valve in a closed position, in accordance with one embodiment of the current invention;

FIG. 3b is a longitudinal cross-sectional view of a portion of a plug valve in an open position, in accordance with one embodiment of the current invention;

FIG. 4 is a longitudinal cross-sectional view of an axial insertion block, in accordance with one embodiment of the current invention;

FIG. 5 is a perspective view of an axial insertion block, in accordance with one embodiment of the current invention;

FIG. 6 is an illustrative perspective view of an inflation adaptor including an axial insertion block, in accordance with one embodiment of the current invention; and

FIG. 7 is a flow diagram of a method for operating an inflation adaptor, in accordance with one embodiment of the current invention.

### DETAILED DESCRIPTION OF THE PRESENTLY PREFERRED EMBODIMENTS

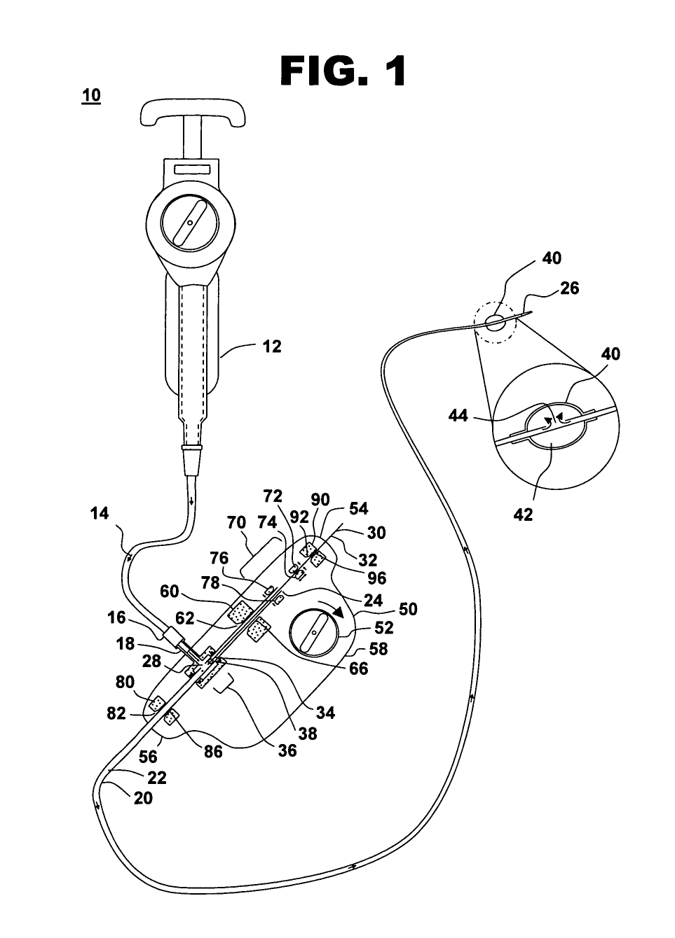

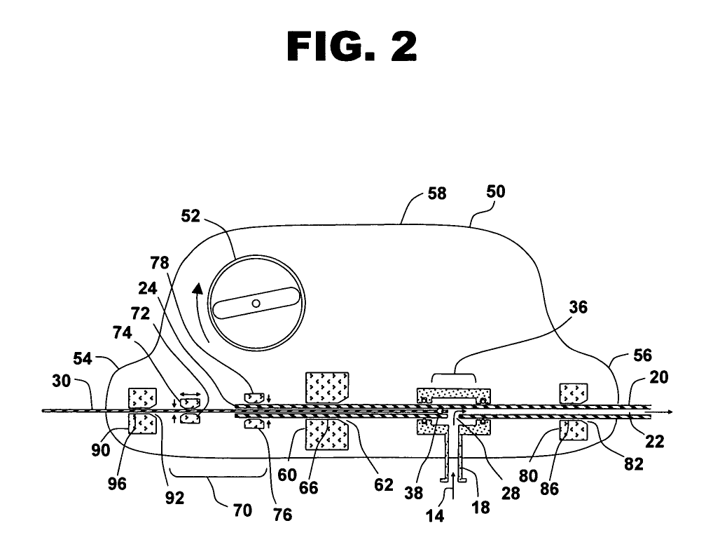

FIGS. 1 and 2 illustrate a system for treating a vascular condition, in accordance with one embodiment of the present invention. Vascular condition treatment system 10 includes hollow guidewire 20 having central lumen 22, core wire 30, inflatable balloon 40, and detachable inflation adaptor 50 with axial insertion block 60 and clamping device 70. Inflatable balloon 40 attached proximate to distal end 26 of hollow guidewire 20 may be inflated with Inflation fluid 14 from inflation fluid supply 12, with transfer of inflation fluid 14 to inflate and deflate inflatable balloon 40 controlled in part by inflation adaptor 50.

For various medical procedures, inflatable balloon 40 comprises, for example, an occlusion balloon, an angioplasty balloon, or a stent-deployment balloon. Vascular condition treatment system 10 may be used, for example, as a temporary occlusion device for blocking fluid flow through arteries or veins. In another medical procedure, vascular condition treatment system 10 is used as a dilation catheter whereby blood vessels with stenoses may be enlarged by inflating inflatable balloon 40 within the blockage. In other applications, vascular condition treatment system 10 is used in coordination with other treatment catheters, such as a stent-delivery catheter, an aspiration catheter, an inspection catheter, a measurement catheter, an angioplasty catheter, an atherectomy catheter, a drug-delivery catheter, an ultrasound device, a measurement device, a laser catheter, an imaging catheter, a treatment catheter or a therapy catheter. The treatment of vascular conditions may include the prevention or correction of various ailments and deficiencies associated with the cardiovascular system, the cerebrovascular system, urogenital systems, biliary conduits, abdominal passageways and other biological vessels within the body.

Inflation fluid supply 12 with inflation fluid 14 may be coupled to inflation adaptor 50 with suitable fitting 16 connected to inflation fluid supply port 18 of inflation adaptor 50. Inflation fluid supply 12 may be coupled to inflation fluid supply port 18 with a removable fluid fitting such as a Luer fitting. Inflation fluid 14 may be a saline solution, a contrast fluid, a dilute contrast agent, or other suitable liquid that is injectable into inflatable balloon 40.

Hollow guidewire 20 is an elongate, flexible tubular member that is inserted into the body to allow treatment of various vascular conditions and obstructions in blood vessels such as atherosclerosis, thrombosis and restenosis. Hollow guidewire 20 may be formed, for example, from an extruded or welded tubular material such as nitinol, stainless steel, or other suitable tubing material. In one example, hollow guidewire 20 has an outer diameter of about 0.014 inches and an inner diameter on the order of 0.009 inches, with a length between 135 centimeters and 300 centimeters. The length of hollow guidewire 20 may be on the order of 300 centimeters, allowing over-the-wire (OTW) catheters to be inserted into the body once hollow guidewire 20 is in place. In another example, hollow guidewire 20 may be on the order of 175 centimeters in length, suitable for guiding treatment catheters of the rapid-exchange, telescope, multi-exchange and/or zipper types, as are known to those of skill in the art.

Central lumen 22 within hollow guidewire 20 allows passage of inflation fluid 14 between inflation adaptor 50 and inflatable balloon 40. Hollow guidewire 20 has proximal end 24 and distal end 26. Inflation hole 28 through sidewall of hollow guidewire 20 near proximal end 24 allows inflation fluid 14 to flow from attached inflation adaptor 50 through central lumen 22 of hollow guidewire 20 to inflatable balloon 40. Inflation hole 28 is positioned to fluidly communicate with inflation fluid supply port 18 while a fluid-tight seal contacts the entire circumference of portions of hollow guidewire 20 distal to and proximal to inflation hole 28. Inflation fluid 14 is injected into interior region 42 of inflatable balloon 40 through balloon inflation hole 44 in sidewall of hollow guidewire 20. Balloon inflation hole 44 may include, for example, one or more holes or apertures formed in the sidewall of hollow guidewire 20, one or more slits in the sidewall, or a spiraling slot cut into the sidewall. At distal end 26 of hollow guidewire 20, central lumen 22 of hollow guidewire 20 may be plugged or capped to prevent fluid from flowing through distal end 26. Additional structures may be added to distal end 26 of hollow guidewire 20, such as a metallic coil or other flexible tubular element, to assist in guiding hollow guidewire 20 through the body. Radiopaque markers and other indicia for determining the location of inflatable balloon 40 may also be added onto hollow guidewire 20.

Core wire 30 comprises a small diameter, flexible wire slidably received within central lumen 22 of hollow guidewire 20. Extended portion 32 of core wire 30 extends outwardly from proximal end 24 of hollow guidewire 20. Axially received portion 34 of core wire 30 is positioned within central lumen 22 of hollow guidewire 20 near proximal end 24. Core wire 30 comprises, for example, a small-diameter wire of stainless steel, nitinol, or other suitably flexible and strong material. Core wire 30 may comprise a polymeric material such as nylon or Teflon@, which has good flexibility and sealing properties yet has sufficient rigidity to controllably translate core wire 30 and valve plug 38, described below, within hollow guidewire 20. A portion of core wire 30 received within hollow guidewire 20, for example, may be sinusoidally shaped to provide a desired degree of friction between core wire 30 and an interior surface of hollow guidewire 20.

Exemplary plug valve 36 includes valve plug 38 attached to axially received portion 34 of core wire 30. Plug valve 36 may be in an open position or a closed position when core wire 30 and hollow guidewire 20 are translated relative to each other. The position of valve plug 38 with respect to inflation hole 18 controls the flow of inflation fluid 14 into and out of inflatable balloon 40. Valve plug 38 has an interference fit with an interior surface of hollow guidewire 20 to provide a fluid seal. Valve plug 38 is formed from a polymeric material such as polyurethane, an epoxy, a silicone, or a semi-compliant polymer with good sealing and wear-resistant properties. In a first valve configuration, valve plug 38 is positioned distal to inflation hole 28 of hollow guidewire 20, and plug valve 36 is in a closed position such that fluid flow is blocked. In a second valve configuration, valve plug 38 is positioned proximal to inflation hole 28 of hollow guidewire 20, and plug valve 36 is in an open position such that fluid flow is allowed. Extended portion 32 of core wire 30 and valve plug 38 are translated relative to hollow guidewire 20 to control a flow of inflation fluid 14 into inflatable balloon 40 by blocking or allowing flow through central lumen 22 of hollow guidewire 20. Pushing core wire 30 into hollow guidewire 20 past inflation hole 28 prevents fluid from flowing into or out of inflatable balloon 40, allowing inflatable balloon 40 to remain inflated when inflation adaptor 50 is removed to allow other treatment catheters to be positioned over hollow guidewire 20 and guided to a treatment location. Re-attaching inflation adaptor 50 and pulling core wire 30 so that valve plug 38 is positioned proximal to inflation hole 28 allows fluid in inflatable region 42 to be removed and inflatable balloon 40 to be deflated. When inflatable balloon 40 is deflated, valve plug 38 may be placed in the closed position to retain some fluid within central lumen 22, and to avoid air and other gases from entering into hollow guidewire 20.

To attach inflation adaptor 50, extended portion 32 of core wire 30 is inserted at distal end 56 of inflation adaptor 50 and is threaded through axial insertion block 60. Axial insertion block 60 is positioned within housing 58 of inflation adaptor 50 to receive core wire portion 32 extending from proximal end 24 of hollow guidewire 20. Core wire portion 32 may extend past proximal end 54 of inflation adaptor 50 when the attachment is complete. Funnel-shaped receiver 62 guides extended portion 32 into wire channel 66 within axial insertion block 60.

When extended portion 32 of core wire 30 is threaded through axial insertion block 60, clamping device 70 may be used for engaging core wire 30. Clamping device 70 is axially aligned with receptor 62 of axial insertion block 60. Clamping device 70 includes a first set of frictional pads or jaws 72 and 74 that engages extended portion 32 of core wire 30 and a second set of jaws 76 and 78 that engages hollow guidewire 20 when clamping device 70 is in a clamped position. Jaws 72, 74, 76 and 78 may have teeth, protrusions, texture, or other features to enhance the gripping of core wire 30 and hollow guidewire 20. Clamping device 70 allows core wire 30 and hollow guidewire 20 to axially move or translate relative to each other when clamping device 70 is in a clamped position to control the flow of inflation fluid 14 into inflatable balloon 40.

Rotation of a multi-position actuation knob 52 coupled to clamping device 70 allow the insertion, engaging, and actuation of plug valve 36 to control flow of inflation fluid 14 into inflatable balloon 40. In a first position, actuation knob 52 allows insertion of core wire extended portion 32 and hollow guidewire proximal end 24 into clamping device 70. In a second position, actuation knob 52 activates clamping device 70 to engage core wire extended portion 32 and hollow guidewire 20. In a third position, actuation knob 52 translates core wire 30 relative to hollow guidewire 20 to control the flow of inflation fluid 14 into and out of inflatable balloon 40.

Inflation fluid 14 from inflation fluid supply 12 coupled to inflation adaptor 50 is injected through a portion of hollow guidewire 20 into interior region 42 of inflatable balloon 40 when clamping device 70 is in a clamped position and when plug valve 36 within hollow guidewire 20 is in an open position. Similarly, inflation fluid 14 may be removed from interior region 42 of inflatable balloon 40 when clamping device 70 is in a clamped position and plug valve 36 is in an open position to deflate inflatable balloon 40.

A distal insertion block 80 including funnel-shaped receiver 82 may be located near distal end 56 to guide core wire extended portion 32 into inflation adaptor 50 and in the approach of portion 32 to axial insertion block 60. Receptor 82 slidably directs extended portion 32 into wire channel 86 within distal insertion block 80 when hollow guidewire 20 with core wire 30 is inserted into inflation adaptor 50. A proximal insertion block 90 including funnel-shaped receiver 92 may be located near proximal end 54 of inflation adaptor 50 to guide extended portion 32 after it has been insertion through axial insertion block 60.

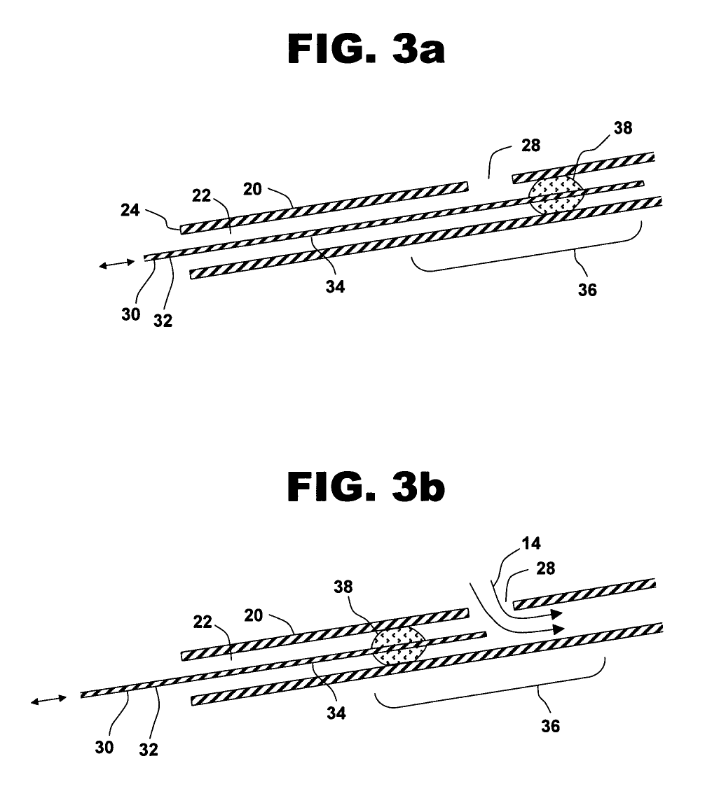

FIG. 3a shows a longitudinal cross-sectional view of a portion of a plug valve in a closed position, in accordance with one embodiment of the present invention. In this figure and following figures, like-numbered elements refer to the same or similar elements as in FIG. 1. Plug valve 36 includes extended portion 32 of core wire 30 extending outwardly from proximal end 24 of hollow guidewire 20. Axially received portion 34 of core wire 30 extends into a portion of central lumen 22 near proximal end 24 of hollow guidewire 20. Plug valve 36 is positioned in one of an open position or a closed position to control the flow of inflation fluid in central lumen 22 of hollow guidewire 20. Axially translating core wire 30 of plug valve 36 controls flow of inflation fluid through central lumen 22 of hollow guidewire 20. Valve plug 38 is attached near a distal end of core wire 30. Valve plug 38 allows inflation fluid to flow in and out of central lumen 22 of hollow guidewire 20. When core wire 30 is translated within hollow guidewire 20 relative to inflation hole 28 formed in a sidewall of hollow guidewire 20, plug valve 36 may be opened and closed, allowing fluid such as inflation fluid to be injected into and withdrawn from central lumen 22 of hollow guidewire 20. When plug valve 36 is closed as shown in FIG. 3a, the flow of inflation fluid is blocked, for example, to prevent air or liquid from flowing through central lumen 22 to and from a distal end of hollow guidewire 20, or to keep an occlusion balloon inflated while in the body. When valve plug 38 is positioned across or distal to inflation hole 28, plug valve 36 is closed. When valve plug 38 is positioned proximal to inflation hole 28 as shown in FIG. 2b, plug valve 36 is open and fluid may flow through inflation hole 28 and central lumen 22 to and from a distal end of hollow guidewire 20.

FIG. 3b shows a longitudinal cross-sectional view of the plug valve of FIG. 3a in an open position, in accordance with one embodiment of the present invention. As core wire 30 is translated to axially move valve plug 38 to a position proximal to inflation hole 28 and to open plug valve 36, inflation fluid 14 may be injected through inflation hole 28 and into central lumen 22 of hollow guidewire 20. Similarly, inflation fluid 14 may be withdrawn from central lumen 22 of hollow guidewire 20 through open inflation hole 28.

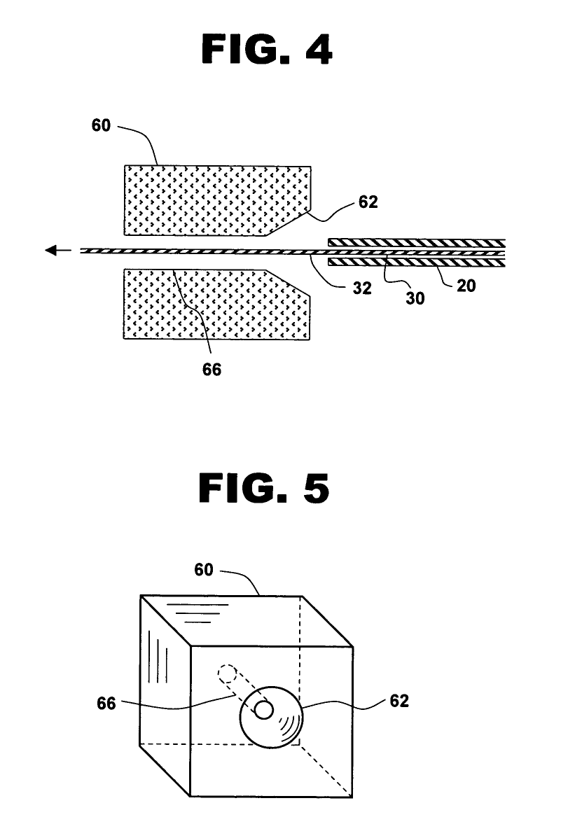

FIG. 4 shows a cross-sectional view of an axial insertion block, in accordance with one embodiment of the present invention. Axial insertion block 60 includes funnel-shaped receiver 62 formed on one side of axial insertion block 60. Receiver 62 guides an insertion of extended portion 32 of core wire 30 into wire channel 66 through axial insertion block 60. Wire channel 66 is appropriately sized to allow the ready insertion of extended core wire 30 and a portion of hollow guidewire 20, while maintaining hollow guidewire 20 in a position suitable for clamping.

FIG. 5 shows a perspective view of an axial insertion block, in accordance with one embodiment of the present invention. Axial insertion block 60 includes funnel-shaped receiver 62 to guide an insertion of a portion of a core wire that extends from a proximal end of a hollow guidewire into axial insertion block 60 and through wire channel 66. Distal insertion blocks and proximal insertion blocks, when used, are similarly constructed. One example of axial insertion block 60 has a facial dimension of substantially 0.639 inches. Another example of axial insertion block 60 has a height of substantially 0.734 inches, a width of substantially 0.377 inches, a receiver diameter of substantially 0.150 inches, a tapered section length of substantially 0.150 inches, and a wire channel diameter of substantially 0.0189 inches.

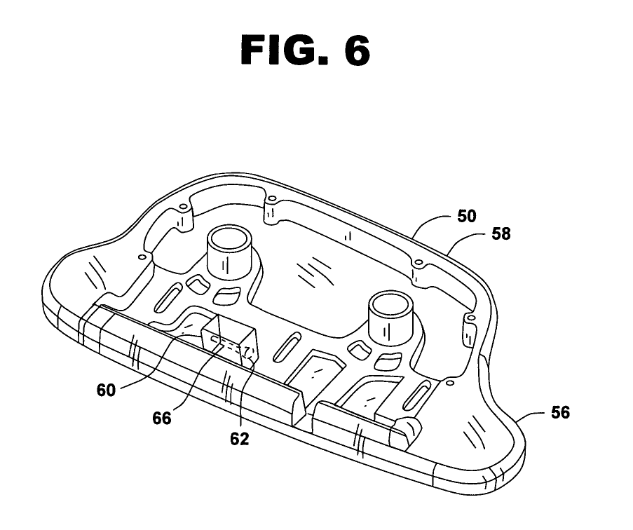

FIG. 6 shows an illustrative perspective view of a component of an inflation adaptor including an axial insertion block, in accordance with one embodiment of the present invention. Axial insertion block 60 is positioned within housing 58 of inflation adaptor 50, with funnel-shaped receiver 62 directed towards distal end 56 of inflation adaptor 50. A hollow guidewire with an extended core wire may be inserted into receiver 62 and guided through wire channel 66. Housing 58 of inflation adaptor 50 is adapted to accommodate and secure axial insertion block 60.

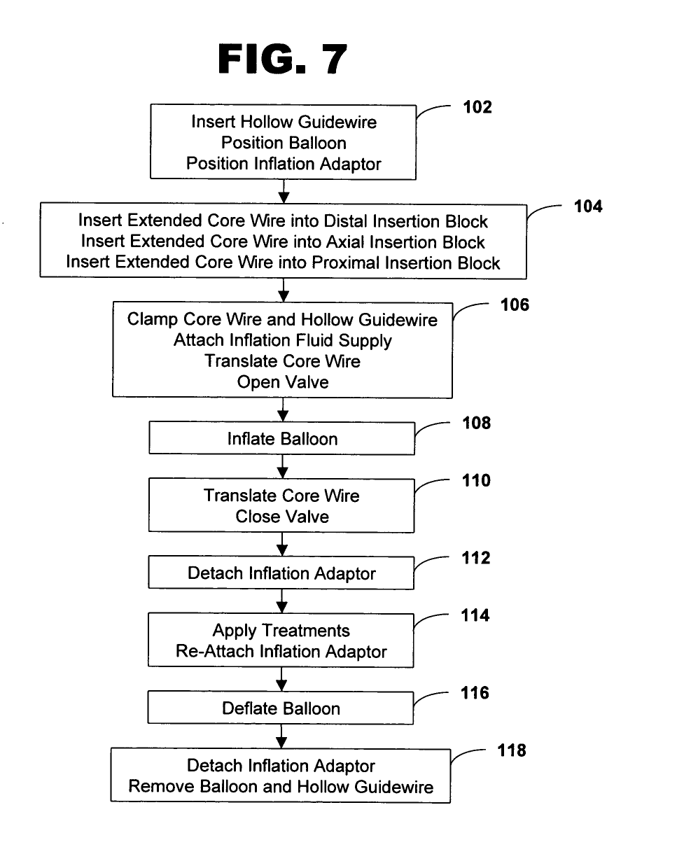

FIG. 7 shows a flow diagram of a method for operating an inflation adaptor, in accordance with one embodiment of the present invention. This method includes various steps to operate an inflation adaptor with an axial insertion block. The description pertains to the inflation and deflation of an inflatable balloon attached near the distal end of a balloon catheter, such as an occlusion catheter. Alternatively, the inflation adaptor operation method may be used to inflate and deflate an angioplasty balloon or to deploy a stent coupled to a stent-deployment balloon. Vascular condition treatment methods employing the inflation adaptor provide, for example, one or more vascular treatments for the prevention or correction of various ailments and deficiencies including those associated with the cardiovascular system, the cerebrovascular system, urogenital systems, biliary conduits, abdominal passageways and other biological vessels within the body.

A balloon catheter with a hollow guidewire and an inflatable balloon is inserted and positioned in a body vessel, as seen at block 102. The hollow guidewire with the inflatable balloon near its distal end is manipulated manually through the vascular system to the desired location for placement of the balloon. For example, a needle puncture is made in the body near the femoral artery, and the hollow guidewire with the inflatable balloon is inserted through the puncture, through the femoral artery, and into a position within a blood vessel where the balloon may be inflated to block fluid flow in the vessel. A central lumen within the hollow guidewire and other lumens may be purged with inflation fluid such as diluted contrast fluid or saline solution before the balloon catheter is inserted into the body. Prior to the positioning of the balloon catheter, fluoroscopic contrast fluid may be injected into the blood vessel in order to identify, visualize and verify the location of a stenosis, blockage, or other medical condition within the blood vessel. In one example, the hollow guidewire and the inflatable balloon are advanced through a vessel and positioned distal to the site of a stenosis.

When the balloon and the hollow guidewire have been appropriately positioned, the inflation adaptor is positioned to receive a core wire partially extended from a proximal end of the hollow guidewire. A portion of the core wire with an attached valve plug extends into a proximal portion of the hollow guidewire, forming a plug valve that controls the flow of fluid through an inflation hole in a sidewall of the hollow guidewire and into the inflatable balloon near the distal end of the hollow guidewire.

To attach the inflation adaptor to the balloon catheter, the extended core wire is inserted into one or more insertion blocks having funnel-shaped receivers, as seen at block 104. For one embodiment of the present invention that has an axial insertion block, a distal insertion block and a proximal insertion block, the proximal end of the extended portion of the core wire is inserted into a funnel-shaped receiver of a distal insertion block before the extended core wire is inserted into a funnel-shaped receiver of an axial insertion block. The extended core wire is then inserted through a funnel-shaped receiver of a proximal insertion block after the extended core wire has been inserted into the axial insertion block. Another embodiment of the present invention, which has neither the distal insertion block nor the proximal insertion block, the extended core wire is inserted into the funnel-shaped receiver of the axial insertion block and through a wire channel in the axial insertion block. The inflation hole in the hollow guidewire is appropriately positioned and aligned with an inflation fluid supply port within the inflation adaptor using, for example, metallic or colored markers, indicia, or other suitable indicators for the inflation hole.

A portion of the extended core wire and the hollow guidewire are clamped, as seen at block 106. For example, a clamping device within the inflation adaptor is actuated by rotating an actuation knob from a first position that allows the insertion of the extended core wire and hollow guidewire to a second position that activates a clamping device to engage the extended core wire and the hollow guidewire. Clamping the extended portion of the core wire and the hollow guidewire comprises, for example, engaging the core wire with a first set of frictional pads or jaws and engaging the hollow guidewire with a second set of jaws. When the core wire and the hollow guidewire are clamped, seals around the inflation hole in the hollow guidewire are secured to allow pressurized fluid from an inflation fluid supply to be injected into the inflation hole and through the hollow guidewire.

Next, an inflation fluid supply is coupled to the inflation adaptor. Inflation fluid such as dilute contrast agent or other suitable fluid may be contained in an inflation device and connected to an inflation fluid port on the inflation adaptor, such as a Luer fitting. Various standard procedures can be used to remove air and other gases from the inflation device, inflation fluid lines connected to the inflation adaptor, and chambers within the inflation adaptor. Alternatively, the inflation fluid supply may be connected to the inflation adaptor prior to attaching the inflation adaptor to the balloon catheter, prior to inserting the hollow guidewire into the body, or at other points depending on the preference of the medical practitioner.

The core wire is translated relative to the hollow guide wire when in the clamped position. Flow of an inflation fluid through a portion of the hollow guidewire is controlled based on the relative translation. For example, a plug valve, which includes a polymeric valve plug attached near a distal end of a portion of the core wire that extends into the hollow guidewire, is operated by axially displacing the core wire with respect to the hollow guidewire. Axial translations of the core wire relative to the hollow guidewire block or allow the flow of inflation fluid through an inflation hole in the side of the hollow guidewire. When in a closed position, the valve plug is located distal to the inflation hole and prevents the flow of fluid through a central lumen of the hollow guidewire. When in an open position, the valve plug is located proximal to the inflation hole, allowing fluid to flow through a portion of the hollow guidewire and into or out from an inflatable balloon near the distal end of the hollow guidewire. The plug valve may be opened, for example, by rotating an actuation knob of the inflation actuator from the second position to a third position, which translates the core wire with respect to the core wire to an open valve position.

The inflatable balloon is inflated, as seen at block 108. The inflatable balloon is inflated by forcing inflation fluid from the inflation fluid supply through a fluid supply port in the inflatable adaptor, through the inflation hole in the sidewall of the hollow guidewire, through the central lumen of the hollow guidewire, through one or more balloon inflation holes of the inflatable balloon, and into an interior region of the inflatable balloon. The inflatable balloon is inflated with the injected inflation fluid to the desired size, which may be monitored with injections of radiopaque contrast fluid and/or with associated x-ray imaging systems. Following the inflation of the balloon, an angiogram using fluoroscopy may be taken to ensure the complete occlusion of a vessel by the balloon.

When the inflatable balloon is inflated to the desired diameter and is occlusively apposed or anchored to the vessel wall, the core wire within the hollow guidewire is axially translated to close the plug valve, as seen at block 110. Translation of the core wire within the hollow guidewire is achieved, for example, by rotating the actuation knob from the third position back to the second position.

Inflation fluid within the inflatable balloon is retained to keep the inflatable balloon inflated and the inflation adaptor is detached, as seen at block 112. The inflation adaptor is detached by unclamping the core wire and hollow guidewire, and then sliding and removing the inflation adaptor from the core wire and hollow guidewire. The core wire and hollow guidewire are unclamped, for example, by rotating the actuation knob from the second position back to the first position.

When the inflation adaptor is detached, one or more treatments may be applied to the vessel, as seen at block 114. In one treatment example, a balloon dilation catheter may be advanced over the hollow guidewire to the treatment site where angioplasty is performed. After the restriction has been treated, the primary treatment catheter may be removed from over the hollow guidewire and then an aspiration catheter can be advanced to the treatment site to aspirate any embolic debris generated during the angioplasty. Once the embolic particles are aspirated, the occlusion balloon is deflated and removed from the patient. Other types of catheters such as imaging catheters, over-the-wire treatment catheters, rapid exchange catheters, stent deployment catheters, inspection catheters or other types of catheters may be used in conjunction with the hollow guidewire.

When one or more treatments have been completed, the inflation adaptor is reattached. The hollow guidewire with the extended core wire is inserted through the receiver(s) of the one or more insertion blocks within the inflation adaptor. After being properly aligned within the inflation adaptor, the hollow guidewire and the extended core wire are clamped. Once clamped, the extended core wire is again translated with respect to the hollow guidewire to slide the valve plug past the inflation hole and open the valve.

The inflatable balloon is deflated, as seen at block 116. The balloon may be deflated when inflation fluid within the inflatable balloon is drawn out or when it is forced out, for example, by elastic restoring forces exerted on the inflation fluid within the interior region by the balloon material. The inflatable balloon may be deflated, for example, with an inflation/deflation device coupled to the inflation adaptor.

Optionally, the inflation adaptor may be detached prior to removal of the hollow guidewire, as seen at block 118. Once the inflatable balloon is deflated, the plug valve is axially translated into a closed position. The inflation fluid supply may be disconnected from the inflation fluid supply port on the inflation adaptor. The hollow guidewire and the core wire are unclamped, and the inflation adaptor is readily removed. The balloon catheter with the hollow guidewire and the inflatable balloon may be repositioned within the body or removed from the body and discarded. When the inflation adapter is used to inflate and deflate the balloon of an angioplasty or stent delivery catheter, steps 110 through 114 are omitted.

In addition to the inflation adaptor being used in treatments employing occlusion balloon catheters, angioplasty catheters, balloon dilation catheters and stent-deployment catheters, it also may be used in other applications such as the deployment of emboli filters and other procedures utilizing controlled axial translations of a wire within a small-diameter hollow tube.

Variations and alterations in the design, manufacture and methods of use of the inflation adaptor are apparent to one skilled in the art, and may be made without departing from the spirit and scope of the present invention. While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A system for treating a vascular condition, comprising:
a hollow guidewire having proximal and distal ends and a central lumen;
a core wire received in the guidewire central lumen and extended from the guidewire proximal end;
an inflatable balloon attached proximate the guidewire distal end;
a detachable inflation adaptor including an axial insertion block having a funnel-shaped receiver to guide the extended core wire into the axial insertion block; and
a clamping device axially aligned with the receiver; wherein the extended core wire and the hollow guidewire are translated relative to each other to control a flow of an inflation fluid into the inflatable balloon while the clamping device is in a clamped position.

2. The system of claim 1 wherein the inflatable balloon comprises one of an occlusion balloon, an angioplasty balloon, and a stent-deployment balloon.

3. The system of claim 1 wherein the clamping device includes first and second sets of jaws that engage the core wire and the hollow guidewire respectively when the clamping device is in a clamped position.

4. The system of claim 1 wherein the clamping device moves the core wire and the hollow guidewire relative to each other when the clamping device is in the clamped position.

5. The system of claim 1 further comprising:
a plug valve having a valve plug attached to a portion of the core wire positioned within the guidewire central lumen, wherein the plug valve is positioned in one of an open position or a closed position when the core wire and the hollow guidewire are translated relative to each other to control the flow of the inflation fluid into the inflatable balloon.

6. The system of claim 1 further comprising:
a multi-position actuation knob coupled to the clamping device, wherein a first position of the actuation knob allows insertion of the extended core wire into the clamping device, and wherein moving the knob from the first position to a second position of the actuation knob activates the clamping device to engage the extended core wire and the hollow guidewire, and wherein moving the knob from the second position to a third position of the actuation knob translates the core wire relative to the hollow guidewire to control the flow of the inflation fluid into the inflatable balloon.

7. The system of claim 1 further comprising:
an inflation fluid supply coupled to the inflation adaptor, wherein the inflation fluid from the inflation fluid supply is injected through a portion of the hollow guidewire into an interior region of the inflatable balloon when the clamping device is in a clamped position and a plug valve within the hollow guidewire is in an open position.

8. The system of claim 1 further comprising:
a distal insertion block having a funnel-shaped receiver to guide the extended core wire into the distal insertion block prior to the insertion of the extended core wire into the axial insertion block.

9. The system of claim 1 further comprising:
a proximal insertion block having a funnel-shaped receiver to guide the extended core wire into the proximal insertion block after the insertion of the extended core wire through the axial insertion block.

10. An inflation adaptor, comprising:
a housing;
an axial insertion block positioned within the housing to receive a core wire extended from a proximal end of a hollow guidewire, the axial insertion block having a funnel-shaped receiver to guide the extended core wire into the axial insertion block; and
a clamping device axially aligned with the receiver; wherein the extended core wire and the hollow guidewire are translated relative to each other to control a flow of an inflation fluid into the hollow guidewire when the clamping device is in a clamped position.

11. A method of operating an inflation adaptor, comprising:
positioning the inflation adaptor to receive a core wire partially extended from a proximal end of a hollow guidewire to define a first valve configuration;
inserting the extended core wire into a funnel-shaped receiver of an axial insertion block disposed within the inflation adaptor;
clamping respective portions of the extended core wire and the hollow guidewire within the inflation adaptor;
relatively translating the core wire and the hollow guidewire to a second valve configuration to control a flow of an inflation fluid through a portion of the hollow guidewire.

12. The method of claim 11 wherein clamping the respective portions of the extended core wire and the hollow guidewire comprises engaging the core wire with a first set of jaws and engaging the hollow guidewire with a second set of jaws.

13. The method of claim 11 wherein the first valve configuration is a closed valve position.

14. The method of claim 11 wherein the second valve configuration is an open valve position.

15. The method of claim 11 further comprising:
inserting the extended core wire into a funnel-shaped receiver of a distal insertion block prior to inserting the extended core wire into the axial insertion block.

16. The method of claim 11 further comprising:
inserting the extended core wire through a funnel-shaped receiver of a proximal insertion block after inserting the extended core wire through the axial insertion block.

17. The method of claim 11 further comprising:
coupling an inflation fluid supply to the inflation adaptor.

18. The method of claim 11 further comprising:
inflating an inflatable balloon attached proximate a distal end of the hollow guidewire.

19. The method of claim 11 further comprising:
deflating an inflatable balloon attached proximate a distal end of the hollow guidewire.
